(19) United States
(12) Patent Application Publication
(10) Pub. No.: US 2026/0144731 A1
(43) Pub. Date: May 28, 2026

(54) AQUEOUS GEL, COSMETIC USE THEREOF AND PROCESS FOR REDUCING THE TACKINESS FELT DURING ITS APPLICATION

(71) Applicant: SOCIETE D'EXPLOITATION DE PRODUITS POUR LES INDUSTRIES CHIMIQUES SEPPIC, Paris Cedex (FR)

(72) Inventors: Sophie Cambos, Castres (FR); Alicia Roso, Castres (FR); Florian Laube, Castres (FR)

(73) Assignee: SOCIETE D'EXPLOITATION DE PRODUITS POUR LES INDUSTRIES CHIMIQUES SEPPIC, Paris Cedex (FR)

(21) Appl. No.: 19/401,006

(22) Filed: Nov. 25, 2025

(30) Foreign Application Priority Data
Nov. 25, 2024 (FR) 2412950

(51) Int. Cl. A61K 8/60; A61K 8/04; A61K 8/34; A61K 8/81; A61K 8/92; A61Q 19/00

(52) U.S. Cl. CPC A61K 8/604; A61K 8/042; A61K 8/342; A61K 8/817; A61K 8/922; A61Q 19/00

(57) ABSTRACT

Aqueous gel comprising from 0.5% by mass to 5.0% by mass of a crosslinked anionic polyelectrolyte (AP) comprising from 5.0 mol % to 95.0 mol % of monomer units derived from 2-methyl-2-((1-oxo-2-propenyl)amino)-1-propanesulfonic acid partially or totally salified in the form of an alkaline salt or an ammonium salt, from 4.9 mol % to 90.0 mol % of monomer units of N,N-dialkylacrylamide and from 0.1 mol % to 5.0 mol % of monomer units derived from a monomer of formula CH₂═C(CH₃)—C(═O)—O—(CH₂—CH₂—O—)_{N}—R₅; from 0.5% by mass to 9.5% by mass of either an alkanol of formula R₁—OH, or a mixture of alkanols of formula R₁—OH; or a composition (C) of alkyl polyglycosides represented by the formula R₁—O-(G)ₓ-H, in which G represents either the glucose residue or the xylose residue and x is a decimal number between 1.05 and 2.05; or a mixture (M) of said alkanol of formula R₁—OH or of said mixture of alkanols of formula R₁—OH and said composition (C); from 0.0% by mass to 3.0% by mass of one or more cosmetic active agents and the amount of water necessary to make up to 100% by mass in which the ratio by mass (R_{M}) having as numerator the mass of the alkanol(s) of formula R₁—OH, or the mass of said composition (C) or the mass of said mixture (M), and as denominator the mass of said crosslinked anionic polyelectrolyte (AP) is greater than or equal to 1 and less than or equal to 10; cosmetic skincare process using said aqueous gel; use either of said alkanol of formula R₁—OH, or a mixture of alkanols of formula R₁—OH, or of said composition (C) or of said mixture (M), with a view to reducing or eliminating the tackiness felt when applying to the skin an aqueous gel comprising said anionic polyelectrolyte (AP); process for reducing or eliminating the tackiness felt during the application to the skin of an aqueous gel comprising said anionic polyelectrolyte (AP) by modifying its composition prior to its application to the skin, by adding either the alkanol(s) of formula R₁—OH, or said composition (C) or said mixture (M).

### TECHNICAL FIELD

The invention relates generally to the field of cosmetics and more particularly to the field of topical cosmetic formulations.

More specifically, the invention relates to improving the sensory properties of aqueous gels. A subject of the invention is thus a novel aqueous gel, optionally rich in salts, the use thereof as a cosmetic composition for topical use, and a novel process for reducing the tackiness of a thickened aqueous gel, optionally rich in salts.

### PRIOR ART

Sera are widely used nowadays for cosmetic treatment of the skin. The term “serum” denotes aqueous solutions which are intended to be absorbed rapidly by the upper layers of the epidermis, and which often contain a high concentration of active ingredients. They are generally used in addition to various skincare formulations to enhance their effect.

Such a treatment comprises a first step of cleansing the skin, then a second step of applying the serum to the cleansed skin and lastly a final step of applying the desired cosmetic care formulation. This process is particularly pertinent when the user observes that the effect of the care formulation is reduced, due to external factors such as the climatic or environmental conditions or in the event of a sudden change of state of the skin, for instance its dryness.

Sera comprise substances that have various properties, such as moisturizing, lightening, purifying, anti-ageing, antiwrinkle, energizing, shadow-concealing or antisebum properties.

Cosmetic sera are generally aqueous gels in which the active substances are kept suspended or solubilized so that they are applied directly to the skin. In this way, the potential for interference with other ingredients, such as oils, on the skin is overcome.

In order for sera to be rapidly absorbed by the stratum corneum, they must have a non-greasy texture, and an equilibrium between flow characteristics that are fluid enough to enable rapid spreading over the surface of the skin to be treated; they must also be gelled enough to avoid running at the time of application.

Synthetic polymers and polymers of natural origins are thickening agents for cosmetic aqueous phases which are well known to those skilled in the art and which make it possible to prepare an aqueous gel for topical uses in the cosmetic and pharmaceutical fields.

The synthetic thickening polymers currently used in this field are in powder form or in liquid form as a water-in-oil emulsion of the polymer prepared by inverse emulsion radical polymerization using surfactants, commonly known as inverse latices. Among the synthetic thickening polymers in powder form that are the most known, mention may be made of polymers based on acrylic acid or esters thereof such as those sold under the names Carbopol™ or Pemulen™. They are notably described in the United States patents U.S. Pat. Nos. 5,373,044 and 2,798,053 and also in the European patent application EP 0 301 532 A2. Mention may also be made of polymers based on 2-acrylamido-2-methylpropanesulfonic acid and salts thereof, such as those sold under the name Aristoflex™. They are notably described in the European patents EP 0 816 403, EP 1 116 733 and EP 1 069 142. These powders are generally obtained by precipitating polymerization of the monomer(s) in solution in an organic solvent of benzene, ethyl acetate, cyclohexane or tert-butanol type; this process thus requires numerous successive stages of purification of the final product, in order to remove any trace of residual solvent.

Among the inverse latices that are the most known, mention may be made of those sold under the names Sepigel™, Simulgel™, Sepiplus™ and Sepilife™. These thickeners are obtained by inverse emulsion radical polymerization. They are easier to handle, notably at room temperature, and disperse very rapidly in water. Furthermore, they develop remarkably high thickening performance qualities. However, as they contain at least one oil, this makes them potentially less suitable for preparing aqueous gels without the possibility of interference between the active agents and the skin.

Synthetic thickeners also exist which have thickening performance qualities equivalent or superior to those of inverse latices, but have more versatile possibilities for use, in particular due to the absence of any oil phase which may lead to aqueous gels. They are in the form of powders but have dissolution times, and thus an ease of use, comparable to those of products that are in the form of liquids. They are described in the European patent application published under the number EP 1 496 081 A2. They are obtained via standard polymerization techniques, such as dispersed-phase radical polymerization, inverse suspension radical polymerization, combined inverse emulsion or inverse microemulsion radical polymerization followed by separation via various techniques such as precipitation in an intermediary solvent, precipitation in an intermediary solvent followed by optional washing, drying by atomization or by azeotropic dehydration, optionally followed by washing with an astutely chosen solvent. These synthetic thickeners thus combine some of the advantages of synthetic thickeners that are in the form of standard powders (absence of oil, production of lighter-coloured aqueous gels) and the advantages of synthetic thickeners that are in the form of inverse latices (pre-neutralization, high rate of dissolution, noteworthy thickening power and stabilizing properties).

However, the gels obtained with these synthetic thickeners do not have satisfactory stability when the composition is rich in electrolytes, as is often the case for sera comprising, for example, electrolyte-rich plant extracts, or mineral salts.

Linear, branched, or crosslinked terpolymers of at least one monomer having a free, partially salified or totally salified strong acid function, and at least one monomer of the formula CH₂═C(CH₃)—C(═O)—O—(CH₂—CH₂—O—)ₙ—R₂, in which R₂ represents a linear or branched alkyl radical including from 8 to 20 carbon atoms and n represents a number greater than or equal to 1 and less than or equal to 30, are described in the international patent application published under the number WO 2011/030 044.

However, when aqueous gels are prepared using such synthetic thickening terpolymers in the presence of electrolytes, consumers experience a tacky feel after application to the skin. This sensation is unpleasant and requires a waiting time before applying another care product that finalizes the routine. Specifically, in general, the serum is applied before the care cream and, consequently, its application to the skin must not make the skin tacky.

The international patent application published under the number WO 2018/024 962 A1 describes the preparation of aqueous gels comprising crosslinked anionic polyelectrolytes of formula (A), salts, electrolyte species, water and sorbitan esters, such as ethoxylated sorbitan oleates, which notably have low turbidity and high stability on storage at room temperature and at 45° C. However, such aqueous gels show tackiness on the skin which is still not satisfactory and needs to be improved.

Another solution may consist in adding silicone oils to the aqueous gel, but silicone oils are abandoned for the preparation of cosmetic formulations because they are not sufficiently biodegradable.

Another solution consists in combining biodegradable alkanes with the aqueous gel, drawing inspiration from the technical teaching contained in international patent application WO 2018/109 353. However, although their contribution is relevant in the context of oil-in-water emulsions, such alkanes give the aqueous gel an unwanted greasy and oily sensory state.

### DESCRIPTION OF THE INVENTION

It thus appeared necessary to develop an aqueous gel that shows a homogeneous appearance after three months of storage at a temperature of 20° C. and also at 45° C., which does not give a tacky sensation after application to the skin and does not comprise oil to avoid any interference with the upper layers of the epidermis.

This is why, according to a first aspect, the invention relates to an aqueous gel characterized in that it comprises, per 100% of its mass:
from 0.5% by mass to 5.0% by mass of a crosslinked anionic polyelectrolyte (AP) including, per 100 mol %:
   from 5.0 mol % to 95.0 mol % of monomer units derived from 2-methyl-2-((1-oxo-2-propenyl)amino)-1-propanesulfonic acid of formula (I) (CH₂═CH—C(═O)—NH)—C(CH₃)₂—S(═O)₂—OH, partially or totally salified in the form of an alkaline salt or an ammonium salt,
   from 4.9 mol % to 90.0 mol % of monomer units derived from a monomer of formula (II) CH₂═CH—C(═O)—N(R₄)₂, in which R₄ represents a linear or branched alkyl radical containing from 1 to 4 carbon atoms, and
   from 0.1 mol % to 5.0 mol % of monomeric units derived from a monomer of formula (III) CH₂═C(CH₃)—C(═O)—O—(CH₂—CH₂—O—)ₙ—R₅, in which R₅ represents a linear or branched alkyl radical including from 8 to 20 carbon atoms and n represents a number greater than or equal to 1 and less than or equal to 20,
from 0.5% by mass to 9.5% by mass:
   either of an alkanol of formula (IV) R₁—OH, in which R₁ represents a linear or branched alkyl radical including from 7 to 22 carbon atoms, or a mixture of alkanols of formulae (IV);
   or a composition (C) of alkyl polyglycosides represented by the formulae (V) R₁—O-(G)ₓ-H, in which G represents either a glucose residue or a xylose residue and x represents a decimal number between 1.05 and 2.05, said composition (C) consisting, per 100 mol %, of a mixture of a₁ mol % of the compound of formula R₁—O-(G)₁-H, a₂ mol % of the compound of formula
   R₁—O-(G)₂-H, a₃ mol % of the compound of formula R₁—O-(G)₃-H, a₄ mol % of the compound of formula R₁—O-(G)₄-H, and as mol % of the compound of formula R₁—O-(G)₅-H, such that the sum a₁%+a₂%+a₃%+a₄%+a₅% is equal to 100 mol % and that the sum a₁%+2a₂%+3a₃%+4a₄%+5a₅% is equal to 100×;
   or a mixture (M) of said alkanol of formula (IV) or said mixture of alkanols of formula (IV) and said composition (C),
from 0.0% by mass to 3.0% by mass of one or more cosmetic active agents chosen from the group consisting of retinol, vitamin C, niacinamide (vitamin B3), undecylenoylphenylalanine, glycolic acid, azelaic acid, hyaluronic acid, the mixture of xylitol, polyglucosides and 1,4-anhydroxylitol and urea, and
the amount of water necessary to make up to 100% by mass, and in that, within said aqueous gel, the mass ratio (R_{M}) having as numerator either the mass of said alkanol of formula (IV) or of said mixture of alkanols of formula (IV), or the mass of said composition (C) or the mass of said mixture (M) and, as denominator, the mass of said crosslinked anionic polyelectrolyte (AP), is greater than or equal to 1.0 and less than or equal to 10.0.

For the purposes of the present invention, the term “crosslinked anionic polyelectrolyte (AP)” denotes a non-linear crosslinked anionic polyelectrolyte, which is in the form of a three-dimensional network which is insoluble in water but swellable in water and which leads to the production of a chemical gel. In this same context, the crosslinking agent used in the polymerization reaction from which said crosslinked anionic polyelectrolyte (AP) is derived is a diethylene or polyethylene compound, more particularly chosen from ethylene glycol dimethacrylate, tetraallyloxyethane, ethylene glycol diacrylate, diallyl urea, triallylamine, trimethylolpropane triacrylate, methylenebis(acrylamide) or a mixture of these compounds. The crosslinking agent is used in this reaction in a molar proportion expressed relative to the monomers used, greater than or equal to 0.005 mol % and less than or equal to 1.000 mol %, more particularly greater than or equal to 0.010 mol % and less than or equal to 0.500 mol %, and most particularly greater than or equal to 0.010 mol % and less than or equal to 0.250 mol %. Depending on the salt concentration of the aqueous medium constituting said topical cosmetic serum, said topical cosmetic serum generally comprises, per 100% of its mass, from 0.5% by mass to 5.0% by mass of said anionic polyelectrolyte (AP).

According to a first particular embodiment, the aqueous gel as defined above is characterized in that said mass ratio (R_{M}) is greater than or equal to 1.5 and less than or equal to 5.0.

According to another particular embodiment, the aqueous gel as defined above is characterized in that said crosslinked anionic polyelectrolyte (AP) includes, per 100 mol %, from 60.0 mol % to 80.0 mol % of monomer units derived from the acid of formula (III), partially salified in ammonium salt form, from 15.0 mol % to 39.5 mol % of monomer units derived from N,N-dimethylacrylamide, and from 0.5 mol % to 5.0 mol % of monomer units derived from tetraethoxylated lauryl methacrylate. An example of such a crosslinked anionic polyelectrolyte (AP) is Sepimax Zen™ sold by the company SEPPIC, which is a trimethylolpropane triacrylate crosslinked terpolymer comprising, per 100 mol %, 77.4 mol % of monomer units derived from ammonium 2-methyl-2-((1-oxo-2-propenyl)amino)-1-propanesulfonate, 19.2 mol % of monomer units derived from N,N-dimethylacrylamide and 3.4 mol % of monomer units derived from tetraethoxylated lauryl methacrylate.

According to another particular embodiment, the aqueous gel as defined above is characterized in that at least one water-soluble salt chosen from the group consisting of alkali metal salts, alkaline-earth metal salts and transition metal salts is dissolved in the water of which said aqueous gel is made, such that the total concentration in the water of said at least one water-soluble salt is greater than 0.00 mol per litre and less than or equal to 1.00 mol per litre. Examples of water-soluble salts that are more particularly present in said aqueous gel as defined above are those chosen from the group consisting of sodium chloride, calcium chloride, magnesium chloride, ammonium chloride, sodium sulfate, calcium sulfate, magnesium sulfate, zinc sulfate, ammonium sulfate, sodium borate, sodium glycolate, sodium citrate, sodium salicylate, sodium lactate, sodium gluconate, zinc gluconate, manganese gluconate, copper gluconate, sodium aspartate and magnesium aspartate.

According to another particular aspect of the present invention, the aqueous gel as defined above is characterized in that said radical R₁ present in formulae (IV) and (V) represents a branched alkyl radical R_{1A} of formula CH₃—(CH₂)ₚ—CH(CH₃—(CH₂)₍ₚ₋₂₎)—CH₂—, where p is an integer greater than or equal to 3 and less than or equal to 9. According to this particular aspect, said alkanol of formula (IV) is preferably 2-octyldodecan-1-ol of formula (IV_{A}) CH₃—(CH₂)₉—CH(CH₃—(CH₂)₇)—CH₂—OH and said composition (C) is preferably a composition (C_{A}) of alkylpolyxylosides of formula (V_{A}) CH₃—(CH₂)₉—CH(CH₃—(CH₂)₇)—CH₂—O-(G_{A})ₓ-H in which G_{A} represents the xylosyl or α,β-D-xylopyranosyl radical and x represents a decimal number of between 1.05 and 2.05, more particularly between 1.10 and 2.00 and even more particularly between 1.20 and 1.80, said composition (C_{A}) consisting, per 100 mol %, of a mixture of a₁ mol % of the compound of formula CH₃—(CH₂)₉—CH(CH₃—(CH₂)₇)—CH₂—O-(G_{A})₁-H, a₂ mol % of the compound of formula CH₃—(CH₂)₉—CH(CH₃—(CH₂)₇)—CH₂—O-(G_{A})₂-H, a₃ mol % of the compound of formula CH₃—(CH₂)₉—CH(CH₃—(CH₂)₇)—CH₂—O-(G_{A})₃-H, a₄ mol % of the compound of formula CH₃—(CH₂)₉—CH(CH₃—(CH₂)₇)—CH₂—O-(G_{A})₄-H, and as mol % of the compound of formula CH₃—(CH₂)₉—CH(CH₃—(CH₂)₇)—CH₂—O-(G_{A})₅-H, such that the sum a₁%+a₂%+a₃%+a₄%+a₅% is equal to 100 mol % and that the sum a₁%+2a₂%+3a₃%+4a₄%+5a₅% is equal to 100×.

In the context of this preferred aspect, a subject of the invention is most particularly the aqueous gel as defined above, comprising a mixture (M_{A}) consisting, per 100% of its mass, of from 10.0% by mass to 50.0% by mass of said composition (C_{A}) and from 50.0% by mass to 90.0% by mass of said alkanol of formula (IV_{A}), in a proportion such that the mass ratio (R_{MA}) having as numerator the mass of said mixture (M_{A}) and as denominator the mass of said crosslinked anionic polyelectrolyte (AP) is greater than or equal to 1.0 and less than or equal to 10.0 and is more particularly greater than or equal to 1.5 and less than or equal to 5.0. An example of such a mixture (M_{A}) is the composition comprising, per 100% of its mass, 75.0% by mass of 2-octyldodecanol and 25.0% by mass of (2-octyldodecyl)polyxylosides resulting from the reaction in acidic medium of xylose and 2-octyldodecanol, sold by the company SEPPIC under the name Fluidanov™ 20X.

According to another particular aspect, the aqueous gel as defined previously is characterized in that said radical R₁ present in formulae (IV) and (V) represents a linear alkyl radical R_{1B} including from 12 to 22 carbon atoms and most particularly from 7 to 22 carbon atoms.

According to this particular aspect, the aqueous gel as defined above is characterized in that said alkanol of formula (IV) or said mixture of alkanols of formula (IV) is preferably a mixture of alkanols of formula (IV_{B}) R_{1B}—OH in which the radical R_{1B} represents a linear alkyl radical containing from 20 to 22 carbon atoms, and said composition (C) is preferably a composition (C_{B}) of alkylpolyglucosides of formula (V_{B}) R_{1B}—O-(G_{B})ₓ-H, in which the radical R_{1B} is as defined above, G_{B} represents the glucosyl or α,β-D-glucopyranosyl radical and x represents a decimal number of between 1.05 and 2.05, more particularly between 1.10 and 2.00, even more particularly between 1.20 and 1.80, said composition (C_{B}) consisting, per 100 mol %, of a mixture of a₁ mol % of the compound of formula R_{1B}—O-(G_{B})₁-H, a₂ mol % of the compound of formula R_{1B}—O-(G_{B})₂-H, a₃ mol % of the compound of formula R_{1B}—O-(G_{B})₃-H, a₄ mol % of the compound of formula R_{1B}—O-(G_{B})₄-H and as mol % of the compound of formula R_{1B}—O-(G_{B})₅-H, such that the sum a₁%+a₂%+a₃%+a₄%+a₅% is equal to 100 mol % and that the sum a₁%+2a₂%+3a₃%+4a₄%+5a₅% is equal to 100×. In the context of this preferred aspect, a subject of the invention is more particularly the aqueous gel as defined above, characterized in that it comprises a mixture (M_{B}) consisting of, per 100% of its mass, from 50.0% by mass to 90.0% by mass of a mixture of arachidyl alcohol and behenyl alcohol and from 10.0% by mass to 50.0% by mass of a composition (C_{B}) of alkyl polyglucosides resulting from the reaction in acidic medium of glucose and said mixture of behenyl alcohol and arachidyl alcohol, in a proportion such that the mass ratio (R_{MB}), with the mass of said mixture (M_{B}) as the numerator and the mass of said crosslinked anionic polyelectrolyte (AP) as the denominator, is greater than or equal to 1.0 and less than or equal to 10.0, and is more particularly greater than or equal to 1.5 and less than or equal to 5.0. An example of such a mixture (M_{B}) is the composition comprising, per 100% of its mass, 80.0% by mass of an equal-mass mixture of behenyl alcohol and arachidyl alcohol and 20.0% by mass of alkyl polyglucosides resulting from the reaction in acidic medium of glucose and said equal-mass mixture of behenyl alcohol and arachidyl alcohol, sold under the name Montanov™ 202 by the company SEPPIC.

The term “oil” denotes a compound and/or a mixture of compounds that are water-insoluble, and liquid at 25° C., and more particularly:
linear alkanes including from 11 to 19 carbon atoms;
branched alkanes including from 7 to 40 carbon atoms, such as isododecane, isopentadecane, isohexadecane, isoheptadecane, isooctadecane, isononadecane or isoeicosane, or mixtures of some of them such as those mentioned below and identified by their INCI name: C7-8 isoparaffin, C8-9 isoparaffin, C9-11 isoparaffin, C9-12 isoparaffin, C9-13 isoparaffin, C9-14 isoparaffin, C9-16 isoparaffin, C10-11 isoparaffin, C10-12 isoparaffin, C10-13 isoparaffin, C11-12 isoparaffin, C11-13 isoparaffin, C11-14 isoparaffin, C12-14 isoparaffin, C12-20 isoparaffin, C13-14 isoparaffin, C13-16 isoparaffin;
cycloalkanes optionally substituted with one or more linear or branched alkyl radicals;
white mineral oils, such as those sold under the following names: Marcol™ 52, Marcol™ 82, Drakeol™ 6VR, Eolane™ 130, Eolane™ 150;
hemisqualane (or 2,6,10-trimethyldodecane; CAS number: 3891-98-3), squalane (or 2,6,10,15,19,23-hexamethyltetracosane), hydrogenated polyisobutene or hydrogenated polydecene;
mixtures of alkanes including from 15 to 19 carbon atoms, said alkanes being linear alkanes, branched alkanes and cycloalkanes, and more particularly the mixture (Mi) which comprises, per 100% of its mass, a mass proportion of branched alkanes of greater than or equal to 90% and less than or equal to 100%; a mass proportion of linear alkanes of greater than or equal to 0% and less than or equal to 9%, and more particularly less than 5%, and a mass proportion of cycloalkanes of greater than or equal to 0% and less than or equal to 1%, for example the mixtures sold under the name Emogreen™ L15 or Emogreen™ L19; - the fatty alcohol ethers of formula (VIII):
Z1-O—Z2 (VIII), in which Z1 and Z2, which may be identical or different, represent a linear or branched alkyl radical containing from 5 to 18 carbon atoms, for example dioctyl ether, didecyl ether, didodecyl ether, dodecyl octyl ether, dihexadecyl ether, (1,3-dimethylbutyl) tetradecyl ether, (1,3-dimethylbutyl) hexadecyl ether, bis(1,3-dimethylbutyl) ether or dihexyl ether;
   monoesters of fatty acids and of alcohols of formula (IX):
R′4-(C═O)—O—R′5 (IX), in which RV(C═O) represents a saturated or unsaturated, linear or branched acyl radical including from 8 to 24 carbon atoms, and R′5 represents, independently of R′4, a saturated or unsaturated, linear or branched hydrocarbon-based chain including from 1 to 24 carbon atoms, for example methyl laurate, ethyl laurate, propyl laurate, isopropyl laurate, butyl laurate, 2-butyl laurate, hexyl laurate, methyl cocoate, ethyl cocoate, propyl cocoate, isopropyl cocoate, butyl cocoate, 2-butyl cocoate, hexyl cocoate, methyl myristate, ethyl myristate, propyl myristate, isopropyl myristate, butyl myristate, 2-butyl myristate, hexyl myristate, octyl myristate, methyl palmitate, ethyl palmitate, propyl palmitate, isopropyl palmitate, butyl palmitate, 2-butyl palmitate, hexyl palmitate, octyl palmitate, methyl oleate, ethyl oleate, propyl oleate, isopropyl oleate, butyl oleate, 2-butyl oleate, hexyl oleate, octyl oleate, methyl stearate, ethyl stearate, propyl stearate, isopropyl stearate, butyl stearate, 2-butyl stearate, hexyl stearate, octyl stearate, methyl isostearate, ethyl isostearate, propyl isostearate, isopropyl isostearate, butyl isostearate, 2-butyl isostearate, hexyl isostearate, isostearyl isostearate;
   diesters of fatty acids and of glycerol of formula (X) and of formula (XI):
R′6-(C═O)—O—CH₂—CH(OH)—CH₂—O—(C═O)—R′7 (X)
R′8-(C═O)—O—CH₂—CHO—(C═O)—R′9-CH₂—OH (XI), in which formulae (X) and (XI) R′6-(C═O), R′7-(C═O), R′8-(C═O) and R′9-(C═O), which may be identical or different, represent a saturated or unsaturated, linear or branched acyl group including from 8 to 24 carbon atoms;
   fatty acid triesters of glycerol of formula (XII):
R′10-(C═O)—O—CH₂—CHO—(C═O)—R′11-CH₂—O—(C═O)—R′12 (XII), in which R′10-(C═O), R′11-(C═O) and R′12-(C═O), which may be identical or different, represent a saturated or unsaturated, linear or branched acyl group including from 8 to 24 carbon atoms;
   plant oils, such as phytosqualane, sweet almond oil, coconut kernel oil, castor oil, jojoba oil, olive oil, rapeseed oil, groundnut oil, sunflower oil, wheat germ oil, corn germ oil, soybean oil, cottonseed oil, alfalfa oil, poppy seed oil, pumpkin seed oil, evening primrose oil, millet oil, barley oil, rye oil, safflower oil, candlenut oil, passionflower oil, hazelnut oil, palm oil, shea butter, apricot kernel oil, beauty-leaf oil, sisymbrium oil, avocado oil, calendula oil, and oils derived from flowers or vegetables;
   ethoxylated plant oils. A subject of the invention is also a cosmetic skincare process characterized in that it comprises the following successive steps:
a step of cleansing the skin;
followed by a step of applying to the cleansed skin the aqueous gel as defined previously, and
optionally followed by a step of applying the desired cosmetic care formulation to the skin.

A subject of the invention is also the use either of an alkanol of formula (IV) R₁—OH, in which R₁ represents a linear or branched alkyl radical including from 7 to 22 carbon atoms, or a mixture of alkanols of formula (IV); or of a composition (C) of alkyl polyglycosides represented by formula (V) R₁—O-(G)ₓ-H, in which G represents either a glucose residue or a xylose residue and x represents a decimal number between 1.05 and 2.05, more particularly between 1.10 and 2.00, even more particularly between 1.20 and 1.80, said composition (C) consisting, per 100 mol %, of a mixture of a₁ mol % of the compound of formula R₁—O-(G)₁-H, a₂ mol % of the compound of formula R₁—O-(G)₂-H, a₃ mol % of the compound of formula R₁—O-(G)₃-H, a₄ mol % of the compound of formula R₁—O-(G)₄-H and as mol % of the compound of formula R₁—O-(G)₅-H, such that the sum a₁%+a₂%+a₃%+a₄%+a₅% is equal to 100 mol % and that the sum a₁%+2a₂%+3a₃%+4a₄%+5a₅% is equal to 100×, or of a mixture (M) of said alkanol of formula (IV) or said mixture of alkanols of formula (IV) and said composition (C), said use being characterized in that it is for the purpose of reducing or eliminating the tackiness felt during the application to the skin of an aqueous gel comprising, per 100% of its mass, from 0.5% by mass to 5.0% by mass of a crosslinked anionic polyelectrolyte (AP) comprising, per 100 mol %, from 5.0 mol % to 95.0 mol % of monomer units derived from 2-methyl-2-((1-oxo-2-propenyl)amino)-1-propanesulfonic acid of formula (I) (CH₂═CH—C(═O)—NH)—C(CH₃)₂—S(═O)₂—OH, partially or totally salified in the form of an alkali metal salt or an ammonium salt, from 4.9 mol % to 90.0 mol % of monomer units derived from a monomer of formula (II) CH₂═CH—C(═O)—N(R₄)₂ in which R₄ represents a linear or branched alkyl radical including from 1 to 4 carbon atoms, and from 0.1 mol % to 5.0 mol % of monomer units derived from a monomer of formula (111) CH₂═C(CH₃)—C(═O)—O—(CH₂—CH₂—O—)ₙ—R₅, in which R₅ represents a linear or branched alkyl radical including from 8 to 20 carbon atoms and n represents a number greater than or equal to 1 and less than or equal to 20.

The term “tacky effect felt during the application of an aqueous gel to the skin” refers to an effect induced by the adhesion force of said gel on the skin. In the context of the present invention, the measurement of the adhesion force is performed by means of a rheometer, notably the DHR2™ rheometer sold by the company Texas Instruments. The adhesion force of a gel must be less than four newtons, so that the user does not feel this tacky effect during application to their skin.

According to another particular aspect, the use as defined previously is characterized in that said crosslinked anionic polyelectrolyte (AP) includes, per 100 mol %, from 60.0 mol % to 80.0 mol % of monomer units derived from the acid of formula (III), partially salified in ammonium salt form, from 15.0 mol % to 39.5 mol % of monomer units derived from N,N-dimethylacrylamide, and from 0.5 mol % to 5.0 mol % of monomer units derived from tetraethoxylated lauryl methacrylate. An example of such a crosslinked anionic polyelectrolyte (AP) is Sepimax Zen™ sold by the company SEPPIC, which is a trimethylolpropane triacrylate crosslinked terpolymer comprising, per 100 mol %, 77.4 mol % of monomer units derived from ammonium 2-methyl-2-((1-oxo-2-propenyl)amino)-1-propanesulfonate, 19.2 mol % of monomer units derived from N,N-dimethylacrylamide and 3.4 mol % of monomer units derived from tetraethoxylated lauryl methacrylate.

According to another particular aspect, the use as defined above is characterized in that said radical R₁ present in formulae (IV) and (V) represents a branched alkyl radical R_{1A} of formula CH₃—(CH₂)ₚ—CH(CH₃—(CH₂)₍ₚ₋₂₎)—CH₂—, where p is an integer greater than or equal to 3 and less than or equal to 9. According to this particular aspect, said alkanol of formula (IV) is preferably 2-octyldodecan-1-ol of formula (IV_{A}) CH₃—(CH₂)₉—CH(CH₃—(CH₂)₇)—CH₂—OH and said composition (C) is preferably a composition (C_{A}) of alkylpolyxylosides of formula (V_{A}) CH₃—(CH₂)₉—CH(CH₃—(CH₂)₇)—CH₂—O-(G_{A})ₓ-H in which G_{A} represents the xylosyl or α,β-D-xylopyranosyl radical and x represents a decimal number of between 1.05 and 2.05, more particularly between 1.10 and 2.00 and even more particularly between 1.20 and 1.80, said composition (C_{A}) consisting, per 100 mol %, of a mixture of a₁ mol % of the compound of formula CH₃—(CH₂)₉—CH(CH₃—(CH₂)₇)—CH₂—O-(G_{A})₁-H, a₂ mol % of the compound of formula CH₃—(CH₂)₉—CH(CH₃—(CH₂)₇)—CH₂—O-(G_{A})₂-H, a₃ mol % of the compound of formula CH₃—(CH₂)₉—CH(CH₃—(CH₂)₇)—CH₂—O-(G_{A})₃-H, a₄ mol % of the compound of formula CH₃—(CH₂)₉—CH(CH₃—(CH₂)₇)—CH₂—O-(G_{A})₄-H, and as mol % of the compound of formula CH₃—(CH₂)₉—CH(CH₃—(CH₂)₇)—CH₂—O-(G_{A})₅-H, such that the sum a₁%+a₂%+a₃%+a₄%+a₅% is equal to 100 mol % and that the sum a₁%+2a₂%+3a₃%+4a₄%+5a₅% is equal to 100×. In the context of this preferred aspect, a subject of the invention is more particularly the use, as defined above, of a mixture (M_{A}) consisting of, per 100% of its mass, from 10.0% by mass to 50.0% by mass of said composition (C_{A}) and from 50.0% by mass to 90.0% by mass of said alkanol of formula (IV_{A}). An example of such a mixture (M_{A}) is the composition comprising, per 100% of its mass, 75.0% by mass of 2-octyldodecanol and 25.0% by mass of (2-octyldodecyl)polyxylosides, sold by the company SEPPIC under the name Fluidanov™ 20X.

According to another particular aspect, the use as defined previously is characterized in that said radical R₁ present in formulae (IV) and (V) represents a linear alkyl radical R_{1B} including from 7 to 22 carbon atoms and most particularly from 7 to 20 carbon atoms. According to this particular aspect, the aqueous gel as defined above is characterized in that said alkanol of formula (IV) or said mixture of alkanols of formula (IV) is preferably a mixture of alkanols of formula (IV_{B}) R_{1B}—OH in which the radical R_{1B} represents a linear alkyl radical containing from 20 to 22 carbon atoms, and said composition (C) is preferably a composition (C_{B}) of alkylpolyglucosides of formula (V_{B}) R_{1B}—O-(G_{B})ₓ-H, in which the radical R_{1B} is as defined above, G_{B} represents the glucosyl or α,β-D-glucopyranosyl radical and x represents a decimal number of between 1.05 and 2.05, more particularly between 1.10 and 2.00, even more particularly between 1.20 and 1.80, said composition (C_{B}) consisting, per 100 mol %, of a mixture of a₁ mol % of the compound of formula R_{1B}—O-(G_{B})₁-H, a₂ mol % of the compound of formula R_{1B}—O-(G_{B})₂-H, a₃ mol % of the compound of formula R_{1B}—O-(G_{B})₃-H, a₄ mol % of the compound of formula R_{1B}—O-(G_{B})₄-H and as mol % of the compound of formula R_{1B}—O-(G_{B})₅-H, such that the sum a₁%+a₂%+a₃%+a₄%+a₅% is equal to 100 mol % and that the sum a₁%+2a₂%+3a₃%+4a₄%+5a₅% is equal to 100×. In the context of this preferred aspect, a subject of the invention is more particularly the use as defined above, of a mixture (M_{B}) consisting of, per 100% of its mass, from 50.0% by mass to 90.0% by mass of a mixture of arachidyl alcohol and behenyl alcohol and from 10.0% by mass to 50.0% by mass of a composition (C_{B}) of alkyl polyglucosides resulting from the reaction in acidic medium of glucose and said mixture of behenyl alcohol and arachidyl alcohol. An example of such a mixture (M_{B}) as defined above is the composition comprising, per 100% of its mass, 80.0% by mass of an equal-mass mixture of behenyl alcohol and arachidyl alcohol and 20.0% by mass of alkyl polyglucosides resulting from the reaction in acidic medium of glucose and said equal-mass mixture of behenyl alcohol and arachidyl alcohol, sold under the name Montanov™ 202 by the company SEPPIC.

A subject of the invention is also a process for reducing or eliminating the tacky effect felt during the application to the skin of an aqueous gel comprising, per 100% of its mass, from 0.5% by mass to 5.0% by mass of a crosslinked anionic polyelectrolyte (AP) including, per 100 mol %, from 5.0 mol % to 95.0 mol % of monomer units derived from 2-methyl-2-((1-oxo-2-propenyl)amino)-1-propanesulfonic acid of formula (I) (CH₂═CH—C(═O)—NH)—C(CH₃)₂—S(═O)₂—OH, partially or totally salified as alkali metal salt or ammonium salt, from 4.9 mol % to 90.0 mol % of monomer units derived from a monomer of formula (II) CH₂═CH—C(═O)—N(R₄)₂ in which R₄ is a linear or branched alkyl radical including from 1 to 4 carbon atoms, and from 0.1 mol % to 5.0 mol % of monomer units derived from a monomer of formula (III) CH₂═C(CH₃)—C(═O)—O—(CH₂—CH₂—O—)ₙ—R₅, in which R₅ represents a linear or branched alkyl radical including from 8 to 20 carbon atoms and n represents a number greater than or equal to 1 and less than or equal to 20, characterized in that the composition of said aqueous gel is modified, before application to the skin, by adding thereto either an alkanol of formula (IV) R₁—OH, in which R₁ is a linear or branched alkyl radical including from 8 to 22 carbon atoms, or a mixture of alkanols of formula (IV); or a composition (C) of alkyl polyglycosides represented by formula (V) R₁—O-(G)ₓ-H, in which G is either the glucose residue or the xylose residue and x is a decimal number between 1.05 and 2.05, more particularly between 1.10 and 2.00, even more particularly between 1.20 and 1.80, said composition (C) consisting, per 100 mol %, of a mixture of a₁ mol % of the compound of formula R₁—O-(G)₁-H, a₂ mol % of the compound of formula R₁—O-(G)₂-H, a₃ mol % of the compound of formula R₁—O-(G)₃-H, a₄ mol % of the compound of formula R₁—O-(G)₄-H and as mol % of the compound of formula R₁—O-(G)₅-H, such that the sum a₁%+a₂%+a₃%+a₄%+a₅% is equal to 100 mol % and that the sum a₁%+2a₂%+3a₃%+4a₄%+5a₅% is equal to 100×; or a mixture (M) of said alkanol of formula (IV) or said mixture of alkanols of formula (IV) and said composition (C), in a proportion such that the mass ratio (R_{M}) in the aqueous gel thus modified, having as numerator either the mass of said alkanol of formula (IV) or said mixture of alkanols of formula (IV), or the mass of said composition (C), or the mass of said mixture (M) and as denominator the mass of said crosslinked anionic polyelectrolyte (AP), is greater than or equal to 1.0 and less than or equal to 10.0 and more particularly greater than or equal to 1.5 and less than or equal to 5.0.

The term “tacky effect felt during the application of an aqueous gel to the skin” refers to an effect induced by the adhesion force of said gel on the skin. In the context of the present invention, the measurement of the adhesion force is performed by means of a rheometer, notably the DHR2™ rheometer sold by the company Texas Instruments. The adhesion force of a gel must be less than four newtons, so that the user does not feel this tacky effect during application to their skin.

According to another particular aspect, the process as defined above is characterized in that said crosslinked anionic polyelectrolyte (AP) includes, per 100 mol %, from 60.0 mol % to 80.0 mol % of monomer units derived from the acid of formula (III), partially salified in ammonium salt form, from 15.0 mol % to 39.5 mol % of monomer units derived from N,N-dimethylacrylamide, and from 0.5 mol % to 5.0 mol % of monomer units derived from tetraethoxylated lauryl methacrylate.

According to another particular aspect, the process as defined above is characterized in that said radical R₁ present in formulae (IV) and (V) represents a branched alkyl radical R_{1A} of formula CH₃—(CH₂)ₚ—CH(CH₃—(CH₂)₍ₚ₋₂₎)—CH₂—, where p is an integer greater than or equal to 3 and less than or equal to 9. According to this particular aspect, said alkanol of formula (IV) is preferably 2-octyldodecan-1-ol of formula (IV_{A}) CH₃—(CH₂)₉—CH(CH₃—(CH₂)₇)—CH₂—OH and said composition (C) is preferably a composition (C_{A}) of alkylpolyxylosides of formula (V_{A}) CH₃—(CH₂)₉—CH(CH₃—(CH₂)₇)—CH₂—O-(G_{A})ₓ-H in which G_{A} represents the xylosyl or α,β-D-xylopyranosyl radical and x represents a decimal number of between 1.05 and 2.05, more particularly between 1.10 and 2.00 and even more particularly between 1.20 and 1.80, said composition (C_{A}) consisting, per 100 mol %, of a mixture of a₁ mol % of the compound of formula CH₃—(CH₂)₉—CH(CH₃—(CH₂)₇)—CH₂—O-(G_{A})₁-H, a₂ mol % of the compound of formula CH₃—(CH₂)₉—CH(CH₃—(CH₂)₇)—CH₂—O-(G_{A})₂-H, a₃ mol % of the compound of formula CH₃—(CH₂)₉—CH(CH₃—(CH₂)₇)—CH₂—O-(G_{A})₃-H, a₄ mol % of the compound of formula CH₃—(CH₂)₉—CH(CH₃—(CH₂)₇)—CH₂—O-(G_{A})₄-H, and as mol % of the compound of formula CH₃—(CH₂)₉—CH(CH₃—(CH₂)₇)—CH₂—O-(G_{A})₅-H, such that the sum a₁%+a₂%+a₃%+a₄%+a₅% is equal to 100 mol % and that the sum a₁%+2a₂%+3a₃%+4a₄%+5a₅% is equal to 100×. In the context of this preferred aspect, a subject of the invention is more particularly the process as defined above, characterized in that the composition of said aqueous gel is modified before its application to the skin by adding thereto a mixture (M_{A}) consisting, per 100% of its mass, of from 10.0% by mass to 50.0% by mass of said composition (C_{A}) and from 50.0% by mass to 90.0% by mass of said alkanol of formula (IV_{A}), in a proportion such that, in the aqueous gel thus modified, the mass ratio (R_{MA}) having as numerator the mass of said mixture (M_{A}) and as denominator the mass of said crosslinked anionic polyelectrolyte (AP) is greater than or equal to 1.0 and less than or equal to 10.0 and is more particularly greater than or equal to 1.5 and less than or equal to 5.0. An example of such a mixture (M_{A}) used in the process as defined above is the composition comprising, per 100% of its mass, 75.0% by mass of 2-octyldodecanol and 25.0% by mass of (2-octyldodecyl)polyxylosides, sold by the company SEPPIC under the name Fluidanov™ 20X.

According to another particular aspect, the process as defined previously is characterized in that said radical R₁ present in formulae (IV) and (V) represents a linear alkyl radical R_{1B} including from 7 to 22 carbon atoms and most particularly from 12 to 22 carbon atoms. According to this particular aspect, the aqueous gel as defined above is characterized in that said alkanol of formula (IV) or said mixture of alkanols of formula (IV) is preferably a mixture of alkanols of formula (IV_{B}) R_{1B}—OH in which the radical R_{1B} represents a linear alkyl radical containing from 20 to 22 carbon atoms, and said composition (C) is preferably a composition (C_{B}) of alkylpolyglucosides of formula (V_{B}) R_{1B}—O-(G_{B})ₓ-H, in which the radical R_{1B} is as defined above, G_{B} represents the glucosyl or α,β-D-glucopyranosyl radical and x represents a decimal number of between 1.05 and 2.05, more particularly between 1.10 and 2.00, even more particularly between 1.20 and 1.80, said composition (C_{B}) consisting, per 100 mol %, of a mixture of a₁ mol % of the compound of formula R_{1B}—O-(G_{B})₁-H, a₂ mol % of the compound of formula R_{1B}—O-(G_{B})₂-H, a₃ mol % of the compound of formula R_{1B}—O-(G_{B})₃-H, a₄ mol % of the compound of formula R_{1B}—O-(G_{B})₄-H and as mol % of the compound of formula R_{1B}—O-(G_{B})₅-H, such that the sum a₁%+a₂%+a₃%+a₄%+a₅% is equal to 100 mol % and that the sum a₁%+2a₂%+3a₃%+4a₄%+5a₅% is equal to 100×. In the context of this preferred aspect, a subject of the invention is more particularly the process as defined above, characterized in that, before its application to the skin, the composition of said aqueous gel is modified by adding thereto a mixture (M_{B}) consisting, per 100% of its mass, of from 50.0% by mass to 90.0% by mass of a mixture of arachidyl alcohol and behenyl alcohol and from 10.0% by mass to 50.0% by mass of an alkyl polyglucoside composition (C_{B}) derived from the reaction in acidic medium of glucose and of said mixture of behenyl alcohol and arachidyl alcohol, in a proportion such that, within said aqueous gel thus modified, the mass ratio (R_{MB}) having as numerator the mass of said mixture (M_{B}) and as denominator the mass of said crosslinked anionic polyelectrolyte (AP) is greater than or equal to 1.0 and less than or equal to 10.0 and is more particularly greater than or equal to 1.5 and less than or equal to 5.0. An example of such a mixture (M_{B}) used in the process as defined above is the composition comprising, per 100% of its mass, 80.0% by mass of an equal-mass mixture of behenyl alcohol and arachidyl alcohol and 20.0% by mass of alkyl polyglucosides resulting from the reaction in acidic medium of glucose and said equal-mass mixture of behenyl alcohol and arachidyl alcohol, sold under the name Montanov™ 202 by the company SEPPIC.

The experimental section that follows illustrates the invention without, however, modifying it.

### Preparation of the Aqueous Qels

The aqueous gels A, A′, B, C, E, E′, F, G, H, L, M, N and P according to the invention and the control gels T1, T′1 and T2 are prepared by mixing their constituents. Their respective compositions are reported in the following Tables 1 to 5, accompanied in Tables 4 and 5 by those of the control solutions T1, T′1 and T2. The preparation process common to all is as follows:
the required amount of water is poured into a reactor equipped with a mechanical stirrer and a jacket comprising a heat-exchange fluid, at a temperature of 20° C., and is then brought to a temperature of 25° C. with mechanical stirring using a Rayneri type blender equipped with a deflocculator, at a speed of 1000 rpm;
the crosslinked anionic polyelectrolyte (AP1) is then gradually added at this temperature of 25° C. with mechanical stirring;
after homogenization of the gel obtained previously, the test anti-tack agent is then gradually added at a temperature of 25° C. with mechanical stirring at a speed of 1000 rpm;
the mixture obtained is then maintained at a temperature of 25° C., and then, if necessary, the salt is added to the medium obtained previously, with continued mechanical stirring.

Gels D, D′, I and J are prepared as above but at a temperature of between 75° C. and 85° C. The optional addition of salt is performed after cooling to 40° C.

TABLE 1

| | Composition of the aqueous gels (mass %) | | | |
|---|---|---|---|---|
| Components ↓ | A | B | C | D |
| Crosslinked anionic | 1.0% | 1.0% | 1.0% | 1.0% |
| polyelectrolyte AP₁ | | | | |
| Fluidanov ™ 20X | 2.0% | 3.0% | 5.0% | 0.0% |
| Arachidyl alcohol | 0.0% | 0.0% | 0.0% | 5.0% |
| 2-Octyldodecanol | 0.0% | 0.0% | 0.0% | 0.0% |
| Water | qs 100% | qs 100% | qs 100% | qs 100% |
| NaCl | 0% | 0% | 0% | 0% |

TABLE 2

| | Composition of the aqueous gels (mass %) | | | |
|---|---|---|---|---|
| Components ↓ | E | A′ | D′ | E′ |
| Crosslinked anionic | 1.0% | 1.0% | 1.0% | 1.0% |
| polyelectrolyte AP₁ | | | | |
| Fluidanov ™ 20X | 0.0% | 2.0% | 0.0% | 0.0% |
| Arachidyl alcohol | 0.0% | 0.0% | 2.0% | 0.0% |
| 2-Octyldodecanol | 5.0% | 0.0% | 0.0% | 2.0% |
| Water | qs 100% | 1.0% | 1.0% | 1.0% |

TABLE 3

| | Composition of the aqueous gels (mass %) | | |
|---|---|---|---|
| Components ↓ | F | G | H |
| Crosslinked anionic | 1.0% | 1.0% | 1.0% |
| polyelectrolyte AP₁ | | | |
| Fluidanox ™ 20X | 2.0% | 0.5% | 1.0% |
| Arachidyl alcohol | 0.0% | 0.0% | 0.0% |
| 2-Octyldodecanol | 0.0% | 0.0% | 0.0% |
| NaCl | 1.0% | 1.0% | 1.0% |
| Water | qs 100% | qs 100% | qs 100% |

TABLE 4

| | Composition of the aqueous gels (mass %) | | | |
|---|---|---|---|---|
| Components ↓ | I | J | T₁ | T₂ |
| Crosslinked anionic | 1.0% | 1.0% | 1.0% | 1.0% |
| polyelectrolyte AP₁ | | | | |
| Fluidanov ™ 20X | 0.0% | 0.0% | 0.0% | 0.0% |
| Myristyl alcohol | 2.0% | 0.0% | 0.0% | 0.0% |
| Montanov ™ 202 | 0.0% | 2.0% | 0.0% | 0.0% |
| NaCl | 1.0% | 1.0% | 0.0% | 1.0% |
| Water | qs 100% | qs 100% | qs 100% | qs 100% |

TABLE 5

| | Composition of the aqueous gels (mass %) ↓ | | | | |
|---|---|---|---|---|---|
| Components ↓ | L | M | N | P | T′₁ |
| Crosslinked anionic | 1.0% | 1.0% | 1.0% | 1.0% | 1.0% |
| polyelectrolyte AP₁ | | | | | |
| 1-Octanol/1-decanol | 2.0% | 0.0% | 0.0% | 0.0% | 0.0% |
| (50/50 by mass) | | | | | |
| Oramix ™CG 110 | 0.0% | 2.0% | 0.0% | 0.0% | 0.0% |
| 1-Heptanol | 0.0% | 0.0% | 2.0% | 0.0% | 0.0% |
| Sepiclear ™ G7 | 0.0% | 0.0% | 0.0% | 2.0% | 0.0% |
| NaCl | 1.0% | 1.0% | 1.0% | 1.0% | 1.0% |
| Water | qs | qs | qs | qs | qs |
| | 100% | 100% | 100% | 100% | 100% |

the crosslinked anionic polyelectrolyte AP1 is a trimethylolpropane triacrylate crosslinked terpolymer comprising, per 100 mol %, 77.4 mol % of monomer units derived from ammonium 2-methyl-2-((1-oxo-2-propenyl)amino)-1-propanesulfonate, 19.2 mol % of monomer units derived from N,N-dimethylacrylamide and 3.4 mol % of monomer units derived from tetraethoxylated lauryl methacrylate.
Fluidanov™ 20X is a composition comprising, per 100% of its mass, 75.0% by mass of 2-octyldodecanol and 25.0% by mass of (2-octyldodecyl)polyxylosides resulting from the reaction in acidic medium of xylose and 2-octyldodecanol;
Montanov™ 202 is a composition comprising, per 100% of its mass, 80.0% by mass of an equal-mass mixture of behenyl alcohol and arachidyl alcohol and 20.0% by mass of alkyl polyglucosides resulting from the reaction in acidic medium of glucose and said equal-mass mixture of behenyl alcohol and arachidyl alcohol.
Oramix™ CG 110 is a composition comprising, per 100% of its mass, 99.0% by mass of a 50/50 mass mixture of 1-octyl polyglucosides and of 1-decyl polyglucosides and 1.0% by mass of water resulting from the reaction in acidic medium of glucose and a 50/50 mass mixture of 1-octanol and 1-decanol.
Sepiclear™ G7 is a composition comprising, per 100% of its mass, 99.0% by mass of n-heptanyl polyglucosides and 1.0% by mass of n-heptan-1-ol.

### Analysis of the Aqueous Gels

The appearance of aqueous gels is assessed visually after seven days of storage at 25° C. and 45° C.;
The viscosities (in MPa·s) of the aqueous gels are measured by means of a Brookfield™ RVT viscometer equipped with a No. 3 spindle at a rotation speed of 6 rpm. Acceptable viscosities are between 5000 mPa·s and 50 000 mPa·s.
The tacky effect is determined by means of a DHR2™ rheometer from the company Texas Instruments, by measuring the adhesion strength of a sample of one 100 μl of aqueous gel on a PMMA plate placed on a Peltier plate, during a rotational stress of the upper mobile plate on the gel, for ten seconds. The normal strength (in Newtons) which holds the upper mobile plate on the Peltier plate (or the adhesion strength) is measured during a succession of ten compression movements followed by rises. Five measurements are taken per gel. The adhesion strength of the gel should be less than 4 newtons, in order for the user not to feel this tacky effect during application to their skin. The results of the analyses are collated in Tables 6 and 7 below:

TABLE 6

| | Aqueous gels ↓ | | | | |
|---|---|---|---|---|---|
| | A′ | E′ | D′ | I | J |
| Appearance at | Homogeneous | | | | |
| 7 days at 25° C. | | | | | |
| Appearance at | Homogeneous | | | | |
| 7 days at 45° C. | | | | | |
| Analyses ↓ | | | | | |
| Viscosity (mPa · s) | 13 200 | 5300 | 4300 | 10 800 | 11 400 |
| Adhesion strength (N) | 3.78 | 3.60 | 3.92 | 3.48 | 3.73 |

TABLE 7

| | Aqueous gels ↓ | | | | |
|---|---|---|---|---|---|
| | F | G | H | T₁ | T₂ |
| Appearance at | Homogeneous | | | | |
| 7 days at 25° C. | | | | | |
| Appearance at | Homogeneous | | | | |
| 7 days at 45° C. | | | | | |
| Analysis ↓ | | | | | |
| Viscosity | 5300 | 5200 | 5000 | 11 200 | 4200 |
| (mPa · s) | | | | | |
| Adhesion | 3.37 | 3.26 | 3.06 | 4.47 | 4.24 |
| strength (N) | | | | | |

TABLE 8

| | Aqueous gels ↓ | | | | |
|---|---|---|---|---|---|
| | L | M | N | P | T′₁ |
| Appearance | Homogeneous | Homogeneous | Homogeneous | Homogeneous | Homogeneous |
| at 7 | | | | | |
| days at | | | | | |
| 25° C. | | | | | |
| Appearance | Homogeneous | Homogeneous | Homogeneous | Homogeneous | Homogeneous |
| at 7 | | | | | |
| days at | | | | | |
| 45° C. | | | | | |
| Analyses ↓ | | | | | |
| Viscosity | 7200 | 1600 | 6600 | 3500 | 6700 |
| (mPa · s) | | | | | |
| Adhesion | 3.43 | 3.21 | 3.17 | 3.18 | 4.58 |
| strength | | | | | |
| (N) | | | | | |

These results demonstrate the decrease in the tackiness induced by the presence, in an aqueous gel thickened with a crosslinked anionic polyelectrolyte, of a fatty alcohol or mixture of fatty alcohols including from 7 to 22 carbon atoms or a composition of fatty alcohols and alkyl polyglycosides in the mass ratio (R_{M}) as claimed.

1. An oil-free aqueous gel comprising, per 100% of its mass:
from 0.5% by mass to 5.0% by mass of a crosslinked anionic polyelectrolyte (AP) including, per 100 mol %:
from 5.0 mol % to 95.0 mol % of monomer units derived from 2-methyl-2-((1-oxo-2-propenyl)amino)-1-propanesulfonic acid of formula (I) (CH₂═CH—C(═O)—NH)—C(CH₃)₂—S(═O)₂—OH, partially or totally salified in the form of an alkaline salt or an ammonium salt,
from 4.9 mol % to 90.0 mol % of monomer units derived from a monomer of formula (II) CH₂═CH—C(═O)—N(R₄)₂, in which R₄ represents a linear or branched alkyl radical containing from 1 to 4 carbon atoms, and
from 0.1 mol % to 5.0 mol % of monomeric units derived from a monomer of formula (III) CH₂═C(CH₃)—C(═O)—O—(CH₂—CH₂—O—)ₙ—R₅, in which R₅ represents a linear or branched alkyl radical including from 8 to 20 carbon atoms and n represents a number greater than or equal to 1 and less than or equal to 20,
from 0.5% by mass to 9.5% by mass:
either of an alkanol of formula (IV) R₁—OH, in which R₁ represents a linear or branched alkyl radical including from 7 to 22 carbon atoms, or a mixture of alkanols of formulae (IV);
or a composition (C) of alkyl polyglycosides represented by the formulae (V) R₁—O-(G)ₓ-H, in which G represents either a glucose residue or a xylose residue and x represents a decimal number between 1.05 and 2.05, the composition (C) consisting, per 100 mol %, of a mixture of a₁ mol % of a compound of formula R₁—O-(G)₁-H, a₂ mol % of the compound of formula R₁—O-(G)₂-H, a₃ mol % of a compound of formula R₁—O-(G)₃-H, a₄ mol % of a compound of formula R₁—O-(G)₄-H, and as mol % of a compound of formula R₁—O-(G)₅-H, such that a sum a₁%+a₂%+a₃%+a₄%+a₅% is equal to 100 mol % and that a sum a₁%+2a₂%+3a₃%+4a₄%+5a₅% is equal to 100×;
or a mixture (M) of the alkanol of formula (IV) or the mixture of alkanols of formula (IV) and the composition (C),
from 0.0% by mass to 3.0% by mass of one or more cosmetic active agents selected from the group consisting of retinol, vitamin C, niacinamide (vitamin B3), undecylenoylphenylalanine, glycolic acid, azelaic acid, hyaluronic acid, a mixture of xylitol, polyglucosides and 1,4-anhydroxylitol, and urea, and
an amount of water necessary to make up to 100% by mass,
wherein, within the aqueous gel, a mass ratio (R_{M}) having as numerator either the mass of the alkanol of formula (IV) or of a mixture of alkanols of formula (IV), or the mass of the composition (C) or the mass of the mixture (M) and, as denominator, the mass of the crosslinked anionic polyelectrolyte (AP), is greater than or equal to 1.0 and less than or equal to 10.0.

2. The aqueous gel according to claim 1, wherein the crosslinked anionic polyelectrolyte (AP) includes, per 100 mol %:
from 60.0 mol % to 80.0 mol % of monomer units derived from an acid of formula (III), partially salified in the form of an ammonium salt,
from 15.0 mol % to 39.5 mol % of monomer units derived from N,N-dimethylacrylamide, and
from 0.5 mol % to 5.0 mol % of monomer units derived from tetraethoxylated lauryl methacrylate.

3. The aqueous gel according to claim 1, wherein at least one water-soluble salt selected from the group consisting of alkali metal salts, alkaline-earth metal salts, and transition metal salts is dissolved in the water of which the aqueous gel is made, such that a total concentration in the water of the at least one water-soluble salt is greater than 0.00 mol per litre and less than or equal to 1.00 mol per litre.

4. The aqueous gel according to claim 1, wherein the alkanol of formula (IV) is 2-octyldodecan-1-ol of formula (IV_{A}) CH₃—(CH₂)₉—CH(CH₃—(CH₂)₇)—CH₂—OH and the composition (C) is a composition (C_{A}) of alkylpolyxylosides of formula (V_{A}) CH₃—(CH₂)₉—CH(CH₃—(CH₂)₇)—CH₂—O-(G_{A})ₓ-H in which G_{A} represents a xylosyl or α,β-D-xylopyranosyl radical and x represents a decimal number of between 1.05 and 2.05, the composition (C_{A}) consisting, per 100 mol %, of a mixture of a₁ mol % of a compound of formula CH₃—(CH₂)₉—CH(CH₃—(CH₂)₇)—CH₂—O-(G_{A})₁-H, a₂ mol % of a compound of formula CH₃—(CH₂)₉—CH(CH₃—(CH₂)₇)—CH₂—O-(G_{A})₂-H, a₃ mol % of a compound of formula CH₃—(CH₂)₉—CH(CH₃—(CH₂)₇)—CH₂—O-(G_{A})₃-H, a₄ mol % of a compound of formula CH₃—(CH₂)₉—CH(CH₃—(CH₂)₇)—CH₂—O-(G_{A})₄-H, and a₅ mol % of a compound of formula CH₃—(CH₂)₉—CH(CH₃—(CH₂)₇)—CH₂—O-(G_{A})₅-H, such that the sum a₁%+a₂%+a₃%+a₄%+a₅% is equal to 100 mol % and that the sum a₁%+2a₂%+3a₃%+4a₄%+5a₅% is equal to 100×.

5. The aqueous gel according to claim 4, wherein the aqueous gel comprises a mixture (M_{A}) consisting, per 100% of its mass, of from 10.0% by mass to 50.0% by mass of the composition (C_{A}) and from 50.0% by mass to 90.0% by mass of an alkanol of formula (IV_{A}), in a proportion such that the mass ratio (R_{MA}) having as numerator the mass of the mixture (M_{A}) and as denominator the mass of the crosslinked anionic polyelectrolyte (AP) is greater than or equal to 1.0 and less than or equal to 10.0.

6. The aqueous gel according to claim 1, characterized in that the alkanol of formula (IV) or the mixture of alkanols of formula (IV) is a mixture of alkanols of formula (IV_{B}) R_{1B}—OH in which a radical R_{1B} represents a linear alkyl radical containing from 20 to 22 carbon atoms, and the composition (C) is a composition (C_{B}) of alkylpolyglucosides of formula (V_{B}) R_{1B}—O-(G_{B})ₓ-H, in which the radical R_{1B} is as defined above, G_{B} represents a glucosyl or α,β-D-glucopyranosyl radical and x represents a decimal number between 1.05 and 2.05, the composition (C_{B}) consisting, per 100 mol %, of a mixture of a₁ mol % of the compound of formula R_{1B}—O-(G_{B})₁-H, a₂ mol % of the compound of formula R_{1B}—O-(G_{B})₂-H, a₃ mol % of the compound of formula R_{1B}—O-(G_{B})₃-H, a₄ mol % of the compound of formula R_{1B}—O-(G_{B})₄-H and a₅ mol % of the compound of formula R_{1B}—O-(G_{B})₅-H, such that the sum a₁%+a₂%+a₃%+a₄%+a₅% is equal to 100 mol % and that the sum a₁%+2a₂%+3a₃%+4a₄%+5a₅% is equal to 100×.

7. The aqueous gel according to claim 6, wherein the aqueous gel comprises a mixture (M_{B}) consisting, per 100% of its mass, of from 50.0% by mass to 90.0% by mass of a mixture of arachidyl alcohol and behenyl alcohol and from 10.0% by mass to 50.0% by mass of an alkyl polyglucoside composition (C_{B}) derived from a reaction in acidic medium of glucose and of the mixture of behenyl alcohol and arachidyl alcohol, in a proportion such that a mass ratio (R_{MB}) having as numerator a mass of the mixture (M_{B}) and as denominator the mass of the crosslinked anionic polyelectrolyte (AP) is greater than or equal to 1.0 and less than or equal to 10.0.

8. A cosmetic skincare process, wherein the process comprises:
cleansing a skin;
applying, to the cleansed skin, the aqueous gel as defined in claim 1, and
optionally, applying a desired cosmetic care formulation to the skin.

9. A process for reducing or eliminating a tacky effect felt, during application to a skin, of an aqueous gel, the process comprising:
providing the aqueous gel comprising, per 100% of its mass,
from 0.5% by mass to 5.0% by mass of a crosslinked anionic polyelectrolyte (AP) including, per 100 mol %,
from 5.0 mol % to 95.0 mol % of monomer units derived from 2-methyl-2-((1-oxo-2-propenyl)amino)-1-propanesulfonic acid of formula (I) (CH2=CH—C(═O)—NH)—C(CH3)2-S(═O)2-OH, partially or totally salified as alkali metal salt or ammonium salt, from 4.9 mol % to 90.0 mol % of monomer units derived from a monomer of formula (II) CH2=CH—C(═O)—N(R4)2 in which R4 is a linear or branched alkyl radical including from 1 to 4 carbon atoms, and
from 0.1 mol % to 5.0 mol % of monomer units derived from a monomer of formula (III) CH2=C(CH3)-C(═O)—O—(CH2-CH2-O-)n-R5, in which R5 represents a linear or branched alkyl radical including from 8 to 20 carbon atoms and n represents a number greater than or equal to 1 and less than or equal to 20,
before applying the aqueous gel to the skin, forming a modified aqueous gel by adding to the aqueous gel:
either an alkanol of formula (IV) R1-OH, in which R1 is a linear or branched alkyl radical including from 8 to 22 carbon atoms, or a mixture of alkanols of formula (IV); or a composition (C) of alkyl polyglycosides represented by formula (V) R1-O-(G)x-H, in which G is either a glucose residue or a xylose residue and x is a decimal number between 1.05 and 2.05, the composition (C) consisting, per 100 mol %, of a mixture of a1 mol % of a compound of formula R1-O-(G)1-H, a2 mol % of the compound of formula R1-O-(G)2-H, a3 mol % of a compound of formula R1-O-(G)3-H, a4 mol % of a compound of formula R1-O-(G)4-H and a5 mol % of a compound of formula R1-O-(G)5-H, such that a sum a1%+a2%+a3%+a4%+a5% is equal to 100 mol % and that a sum a1%+2a2%+3a3%+4a4%+5a5% is equal to 100×; either a mixture (M) of the alkanol of formula (IV) or the mixture of alkanols of formula (IV) and the composition (C), in a proportion such that the mass ratio (RM) in the aqueous gel thus modified, having as numerator either the mass of the alkanol of formula (IV) or the mixture of alcohols of formula (IV), or the mass of the composition (C), or the mass of the mixture (M) and as denominator a mass of the crosslinked anionic polyelectrolyte (AP), is greater than or equal to 1.0 and less than or equal to 10.0 and more particularly greater than or equal to 1.5 and less than or equal to 5.0, and
applying the modified aqueous gel to the skin.

10. The process according to claim 9, wherein the crosslinked anionic polyelectrolyte (AP) includes, per 100 mol %, from 60.0 mol % to 80.0 mol % of monomer units derived from the acid of formula (I), partially salified in ammonium salt form, from 15.0 mol % to 39.5 mol % of monomer units derived from N,N-dimethylacrylamide, and from 0.5 mol % to 5.0 mol % of monomer units derived from tetraethoxylated lauryl methacrylate.

11. The process according to claim 9, wherein the alkanol of formula (IV) is 2-octyldodecan-1-ol of formula (IV_{A}) CH₃—(CH₂)₉—CH(CH₃—(CH₂)₇)—CH₂—OH and the composition (C) is a composition (C_{A}) of alkylpolyxylosides of formula (V_{A}) CH₃—(CH₂)₉—CH(CH₃—(CH₂)₇)—CH₂—O-(G_{A})ₓ-H in which G_{A} represents a xylosyl or α,β-D-xylopyranosyl radical and x represents a decimal number of between 1.05 and 2.05, the composition (C_{A}) consisting, per 100 mol %, of a mixture of a₁ mol % of a compound of formula CH₃—(CH₂)₉—CH(CH₃—(CH₂)₇)—CH₂—O-(G_{A})₁-H, a₂ mol % of a compound of formula CH₃—(CH₂)₉—CH(CH₃—(CH₂)₇)—CH₂—O-(G_{A})₂-H, a₃ mol % of a compound of formula CH₃—(CH₂)₉—CH(CH₃—(CH₂)₇)—CH₂—O-(G_{A})₃-H, a₄ mol % of a compound of formula CH₃—(CH₂)₉—CH(CH₃—(CH₂)₇)—CH₂—O-(G_{A})₄-H, and a₅ mol % of a compound of formula CH₃—(CH₂)₉—CH(CH₃—(CH₂)₇)—CH₂—O-(G_{A})₅-H, such that the sum a₁%+a₂%+a₃%+a₄%+a₅% is equal to 100 mol % and that the sum a₁%+2a₂%+3a₃%+4a₄%+5a₅% is equal to 100×.

12. The process according to claim 11, wherein the aqueous gel is modified prior to its application to the skin by adding thereto a mixture (M_{A}) consisting, per 100% of its mass, of from 10.0% by mass to 50.0% by mass of the composition (C_{A}) and from 50.0% by mass to 90.0% by mass of the alkanol of formula (IV_{A}), in a proportion such that, within the aqueous gel thus modified, the mass ratio (R_{MA}) having as numerator the mass of the mixture (M_{A}) and as denominator the mass of the crosslinked anionic polyelectrolyte (AP) is greater than or equal to 1.0 and less than or equal to 10.0 and is more particularly greater than or equal to 1.5 and less than or equal to 5.0.

13. The process according to claim 9, wherein the alkanol of formula (IV) or the mixture of alkanols of formula (IV) is a mixture of alkanols of formula (IV_{B}) R_{1B}—OH in which the radical R_{1B} represents a linear alkyl radical containing from 20 to 22 carbon atoms, and the composition (C) is a composition (C_{B}) of alkylpolyglucosides of formula (V_{B}) R_{1B}—O-(G_{B})ₓ-H, in which a radical R_{1B} is as defined above, G_{B} represents a glucosyl radical or α,β-D-glucopyranosyl and x represents a decimal number of between 1.05 and 2.05, the composition (C_{B}) consisting, per 100 mol %, of a mixture of a₁ mol % of a compound of formula R_{1B}—O-(G_{B})₁-H, a₂ mol % of the compound of formula R_{1B}—O-(G_{B})₂-H, a₃ mol % of a compound of formula R_{1B}—O-(G_{B})₃-H, a₄ mol % of a compound of formula R_{1B}—O-(G_{B})₄-H and a₅ mol % of a compound of formula R_{1B}—O-(G_{B})₅-H, such that the sum a₁%+a₂%+a₃%+a₄%+a₅% is equal to 100 mol % and that the sum a₁%+2a₂%+3a₃%+4a₄%+5a₅% is equal to 100×.

14. The process according to claim 13, wherein, before its application to the skin, the aqueous gel is modified by adding thereto a mixture (M_{B}) consisting, per 100% of its mass, of from 50.0% by mass to 90.0% by mass of a mixture of arachidyl alcohol and behenyl alcohol and from 10.0% by mass to 50.0% by mass of an alkyl polyglucoside composition (C_{B}) derived from a reaction in acidic medium of glucose and of the mixture of behenyl alcohol and arachidyl alcohol, in a proportion such that, within the aqueous gel thus modified, a mass ratio (R_{MB}) having as numerator a mass of the mixture (M_{B}) and as denominator a mass of the crosslinked anionic polyelectrolyte (AP) is greater than or equal to 1 and less than or equal to 10.

15. The process according to claim 13, wherein, before its application to the skin, the aqueous gel is modified by adding thereto a mixture (M_{B}) consisting, per 100% of its mass, of from 50.0% by mass to 90.0% by mass of a mixture of arachidyl alcohol and behenyl alcohol and from 10.0% by mass to 50.0% by mass of an alkyl polyglucoside composition (C_{B}) derived from a reaction in acidic medium of glucose and of the mixture of behenyl alcohol and arachidyl alcohol, in a proportion such that, within the aqueous gel thus modified, a mass ratio (R_{MB}) having as numerator a mass of the mixture (M_{B}) and as denominator a mass of the crosslinked anionic polyelectrolyte (AP) is greater than or equal to 1.5 and less than or equal to 5.0.
